(19) United States
(12) Patent Application Publication
(10) Pub. No.: US 2026/0263754 A1
(43) Pub. Date: Sep. 10, 2026

(54) GUIDEWIRE SYSTEM

(71) Applicant: MicroVention, Inc., Aliso Viejo, CA (US)

(72) Inventors: Belete Degaga, Aliso Viejo, CA (US); Joseph Gulachenski, Aliso Viejo, CA (US); Nicholas Davis, Aliso Viejo, CA (US); Tadele Wolde-Meskel, Aliso Viejo, CA (US); Barry Tan, Aliso Viejo, CA (US); Abraham Avila, Aliso Viejo, CA (US)

(73) Assignee: MicroVention, Inc., Aliso Viejo, CA (US)

(21) Appl. No.: 19/135,016

(22) PCT Filed: Dec. 7, 2023
(86) PCT No.: PCT/US2023/082954

§ 371 Date: Jun. 2, 2025

Related U.S. Application Data
(60) Provisional application No. 63/386,422, filed on Dec. 7, 2022.

(51) Int. Cl. A61M 25/09

(52) U.S. Cl. CPC A61M 25/09; A61M 2025/09083; A61M 2025/09166; A61M 2025/09175

(57) ABSTRACT

A guidewire system having improved torque ability and shapeability for traversing tortuous vasculatures to reach a distant anatomy. The guidewire system may include a core wire having a proximal portion and a distal portion. The distal portion may be tapered along at least a portion of its length. The distal portion may include both a plurality of uniform diameter segments and a plurality of tapered diameter segments. One or more coils may be affixed to the core wire, such as an inner coil and an outer coil. A radiopaque marker may be affixed to a distal tip of the core wire for visualization by an imaging device. Alternatively, one or more coiled grooves may be cut into the core wire to perform the same function as the aforementioned coils. The entirety of the core wire, including any affixed coils, may be encapsulated in a polymer jacket.

### RELATED APPLICATIONS

This application claims priority to U.S. Provisional Application Ser. No. 63/386,422 filed Dec. 7, 2022 entitled Guidewire System, which is hereby incorporated herein by reference in its entirety.

### BACKGROUND

Navigation of various medical devices to distal anatomies through tortuous vasculatures can be incredibly challenging under certain circumstances. For example, guidewire navigation through the cerebral hemisphere may require a significant amount of torqueability, distal tip shape ability, and/or the ability to retain the guidewire's original shape to navigate further into the vasculature.

When navigating under such potentially difficult conditions as tortuous anatomy, it may be desirable for a guidewire to have a shapeable tip which can both aid in efficient introduction into the human body and provide a torqueability advantage. Such an approach may rely upon the location of each crossing profile and/or the stiffness of the core wire of the guidewire as a determining factor for the quality of torqueability of the guidewire.

### SUMMARY

Disclosed herein are example embodiments of a guidewire system which include a core wire having a distal section which is tapered from a larger outer diameter to a smaller outer diameter. In an example embodiment, the distal section may comprise both uniform diameter segments and tapered diameter segments. The uniform and tapered diameter segments may be configured to alternate along a length of the distal section of the core wire.

In an example embodiment, one or more coils may be connected to or position around at least a portion of the distal section of the core wire. In an example embodiment, the one or more coils may comprise an inner coil and an outer coil. In an example embodiment, an additional extension coil may be positioned around or connected to a distal tip of the core wire.

In an example embodiment, the guidewire may have a variable crossing profile and stiffness across a distal section, such as for a distal length of about 40 cm-50 cm.

In an example embodiment, a polymer outer jacket may encapsulate the inner coil, the outer coil, and the core wire.

In an example embodiment, the inner and outer coils may have substantially similar filars.

In an example embodiment, the outer coil may be composed of platinum tungsten and the inner coil may be composed of stainless steel.

In an example embodiment, one or both of the inner and/or outer coils may have a distally increased pitch.

In an example embodiment, the inner coil may comprise distally increased pitch dimensions of about 0.005 inches, 0.008 inches, and 0.013 inches.

In an example embodiment, the inner coil may have about a 15 mm long distally increased pitch.

In an example embodiment, the inner and outer coils may be substantially the same length.

In an example embodiment, soldering may be utilized at the distal and proximal sections of the respective coils to keep them in place with respect to the core wire.

In an example embodiment, a guidewire may allow the entry of a polymer jacket between the coils and the core wire. Thus, a polymer jacket may be applied over the coils and underlying core wire.

In an example embodiment, the polymer jacket may maintain about a 15%-20% mixup ratio.

In an example embodiment, the polymer jacket may be applied over the coils and core wire using a dip coating method.

In an example embodiment, an extension coil may be utilized in addition to the inner and outer coils.

In an example embodiment, a length of the extension coil may be approximately about 8%-11% of a length of the outer coil or the inner coil.

In an example embodiment, the extension coil may be composed of the same material as the outer coil.

In an example embodiment, the extension coil may have a uniform pitch with respect to the inner and/or outer coils.

In an example embodiment, the polymer jacket may also be applied over the extension coil.

In an example embodiment, rather than using discrete coils which are connected to the core wire, the core wire may instead include radial grooves cut into the core wire which provide similar functionality to attached coils. The radial grooves may comprise a first groove which is cut in a sweep pattern in a clockwise direction and a second groove which is cut in a sweep pattern in a counterclockwise direction.

In an example embodiment, vascular access may be enhanced, and better guidewire tracking control may be achieved by transmitting torque efficiently from a proximal end to a distal tip of a guidewire.

In an example embodiment, a guidewire may have a variable crossing profile and stiffness across its distal section, such as the distal 40 cm-50 cm section.

In an example embodiment, a distal tip of a guidewire may be flattened.

In an example embodiment, a guidewire may maintain a minimum annular region between its core wire and an inner coil secured thereto.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. Reference being made to the accompanying drawings, in which

FIG. 1 illustrates a perspective view of a core wire of a guidewire system, in accordance with an example embodiment of the present disclosure.

FIG. 2 illustrates a side view of a core wire of a guidewire system, in accordance with an example embodiment of the present disclosure.

FIG. 3 illustrates a graph of a core wire crossing profile, in accordance with an example embodiment of the present disclosure.

FIG. 4 illustrates the graph of FIG. 3 in table form illustrating taper values of a core wire of a guidewire system, in accordance with an example embodiment of the present disclosure.

FIG. 5 illustrates a side view of a guidewire system, in accordance with an example embodiment of the present disclosure.

FIG. 6 illustrates a side view of a second (outer) coil of a guidewire system, in accordance with an example embodiment of the present disclosure.

FIG. 7 illustrates a side view of a first (inner) coil of a guidewire system, in accordance with an example embodiment of the present disclosure.

FIG. 8 illustrates a side view of a guidewire system where coils are positioned over a portion of the length of the core wire, in accordance with an example embodiment of the present disclosure.

FIG. 9 illustrates a side view of a guidewire system that include three coils, in accordance with an example embodiment of the present disclosure.

FIG. 10 illustrates a side view of a third (extension coil) of a guidewire system, in accordance with an example embodiment of the present disclosure.

FIG. 11 illustrates a side view of a core wire of a guidewire system into which coiled grooves have been cut, in accordance with an example embodiment of the present disclosure.

FIG. 12 illustrates a side view of a distal tip of a core wire of a guidewire system, in accordance with an example embodiment of the present disclosure.

FIG. 13 illustrates a side view of a distal tip of a core wire of a guidewire system, in accordance with an example embodiment of the present disclosure.

FIG. 14 illustrates a sectional view of a distal tip of a core wire of a guidewire system, in accordance with an example embodiment of the present disclosure.

FIG. 15 illustrates of a sweep cut pattern for opposing first and second coiled grooves, in accordance with an example embodiment of the present disclosure.

FIG. 16 illustrates a side view of a guidewire system with a radiopaque marker affixed over a portion of the distal portion of the core wire, in accordance with an example embodiment of the present disclosure.

### DETAILED DESCRIPTION

Specific embodiments of the disclosure will now be described with reference to the accompanying drawings. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. The terminology used in the detailed description of the embodiments illustrated in the accompanying drawings is not intended to be limiting of the disclosure. In the drawings, like numbers refer to like elements.

For the purposes of this specification, use of the terms “about,” “around,” or “approximately” when referring to a value may be understood to mean within 5% of the stated value (either greater or lesser), inclusive.

A guidewire system may comprise a core wire, an inner coil, an outer coil, and/or an extension coil.

The guidewire system may comprise an elongated body including a proximal portion and a distal portion. The elongated body may be comprised of a core wire. The proximal portion of the core wire may be comprised of a uniform diameter and a distal portion of the core wire may be comprised of an overall tapered portion. Thus, the outer diameter of the core wire may reduce between a proximal end of its distal portion and a distal end of its distal portion. The rate of taper may vary across a length of the distal portion (e.g., the taper may not be uniform). The core wire may be ground to form the tapered portion. The core wire may be composed of various materials. For example, the core wire may be composed of Nitinol.

The proximal portion of the core wire may form greater than half of a length of the core wire such that the proximal portion is of a greater length than the distal portion. By way of example, a total length of the core wire may be about 2000 mm, a total length of the proximal portion of the core wire may be about 1600 mm, and a total length of the distal portion of the core wire may be about 400 mm.

The tapered portion may comprise only tapered diameter sections. Each tapered diameter section may reduce in diameter towards a distal end of the elongated body. Multiple tapered diameter sections may be utilized, with differing degrees of taper (e.g., rate of reduction of diameter towards the distal end). Thus, a first tapered section may comprise a first degree or taper and a second tapered section may comprise a second degree of taper which is different (e.g., greater or lesser) than the first degree of taper.

A third tapered section may also be included which may have a third degree of taper, different from the first and second degrees of taper, or may have the first or second degree of taper. Thus, where multiple tapered sections are utilized in the tapered portion, two or more of the tapered sections may share substantially the same degree of taper and one or more of the tapered sections may have a different degree of taper.

The tapered portion may itself be comprised of a plurality of uniform diameter regions and a plurality of variable (e.g., tapered) diameter regions. The uniform and tapered diameter regions may be tapered such that each tapered diameter region has at least one adjacent uniform diameter region, or such that each uniform diameter region has at least one adjacent tapered diameter region. Thus, the tapered and uniform diameter regions of the tapered portion may be staggered.

The lengths of each of the respective uniform and tapered diameter regions may vary across the length of the tapered portion such that, e.g., a first uniform diameter region is not the same length as a second uniform diameter region, a first tapered diameter region is not the same length as a second tapered diameter region, and so on. The combined length of the uniform diameter regions may be greater than the combined length of the tapered diameter regions. By way of example, the combined length of the uniform diameter regions may be about 390 mm and the combined length of the tapered diameter regions of the distal portion may be about 10 mm.

The outer diameters of the core wire across its length may vary. For example, the outer diameter of the proximal portion of the core wire, which may be uniform across its length, may be about 0.0135 inches. The outer diameter of the distal portion of the core wire, which may “step down” across its length, may comprise stepped down values of about 0.008 inches, 0.0069 inches, 0.0055 inches, 0.0047 inches, and 0.0029 inches.

A distal tip of the core wire may be flattened so as to form a paddle. A length of the paddle at the distal end of the core wire may vary. For example, the paddle may have a length of about 5mm-11mm. The paddle may perform a number of functions. For example, the paddle may accommodate both the inner and outer coils. The paddle may maintain a minimum annular region between itself and the inner coil. The paddle may allow the entry of the polymer jacket between the coils and the core wire.

One or more coils may be affixed to the core wire. For example, an inner coil and an outer coil may be affixed to the core wire. The inner coil may be affixed directly over the core wire, and the outer coil may be affixed directly over the inner coil such that the inner coil is nested within the outer coil.

The inner coil may be composed of various materials including stainless steel. The overall length of the inner coil may be comprised of about 250-350 mm. The coil pitch of the inner coil may increase distally. By way of example, there may be about a 0.0005 inch coil gap for a proximal portion of the inner coil, followed by about a 0.00175 inch coil gap at a medial portion of the inner coil. A distal portion of the inner coil, such as a distal 15 mm length of the inner coil, may comprise coil pitches of about 0.0013 inches, 0.008 inches, and 0.005 inches towards a distal tip of the inner coil. Such a configuration may improve shapeability of the distal tip of the guidewire.

The outer coil may be composed of various materials including platinum tungsten. Thus, the material of the inner coil may not be the same as the inner coil. However, in some circumstances, the inner coil and the outer coil may be composed of the same materials. The overall length of the outer coil may be the same as or different than the overall length of the inner coil. By way of example, the overall length of the outer coil may be about 250-350 mm. The inner coil may be slightly longer in length than the outer coil.

Both the inner and outer coils may comprise substantially the same filar outer diameter, such as about 0.00125 inches. The coil pitch of the outer coil may be uniform across its length, such as about 0.00175 inches. The coil gap of the outer coil may be uniform across its length, such as about 0.0005 inches.

The inner and outer coils may have opposite orientations with respect to each other. Thus, the inner coil may be wound in a first direction and the outer coil may be wound in a second direction, with the first direction being opposite to the second direction. Put differently, the inner and outer coils may cross each other at numerous locations along their respective lengths to form a crossing pattern across the length of the core wire to which they are affixed.

To maintain the positioning of the outer and inner coils both with respect to each other and with respect to the core wire, the entirety of the core wire, inner coil, and outer coil may be encapsulated in a polymer jacket. Various polymers known in the art may be utilized. The polymer jacket may maintain about a 15%-20% mix up ratio. The manner by which the polymer jacket is applied may vary. For example, the polymer jacket may be applied by dip coating. The use of such a polymer jacket to cover and encapsulate the core wire, inner coil, and outer coil may function to keep the coils and the core wire intact.

A third, extension coil may also be utilized. The extension coil may be affixed to a distal tip of the core wire. The extension coil may at least partially overlap the inner and/or outer coils or may be axially positioned with respect to the inner and/or outer coils such that a proximal end of the extension coil abuts against or is secured against a distal end of the inner and/or outer coils. The extension coil may rest over the inner and/or outer coils.

The extension coil may be composed of various materials. For example, the extension coil may be composed of platinum tungsten. Thus, the extension coil may be composed of the same material as the outer coil. The extension coil may be shorter in length than the inner and/or outer coils. For example, the extension coil may be between about 20 mm-30 mm in length. As a further example, the extension coil may be between about 6%-12% of a length of the inner and/or outer coils.

The extension coil may have a uniform pitch across its length. However, the pitch of the extension coil may vary across its length in some circumstances. The direction of winding of the extension coil may vary. The extension coil may be wound in the same direction as the inner coil, in which case the extension coil would be wound in an opposite direction as the outer coil. Conversely, the extension coil may be wound in the same direction as the outer coil, in which case the extension would be wound in the opposite direction as the inner coil.

The extension coil may be affixed to surround the distal tip of the core wire. As the distal tip of the core wire is of a smaller diameter than the extension coil, there may be an annular region defined between the extension coil and the distal tip of the core wire. Such an annular region may be useful for introducing the polymer jacker between the respective coils and the core wire to which they are affixed such that the polymer jacket penetrates within the coils. The extension coil may be soldered to the distal tip of the core wire, or otherwise secured thereto using various methods known in the art.

A guidewire system may comprise an elongated body into which one or more grooves are cut to perform a similar function as the aforementioned coil(s). The elongated body may comprise a core wire. The core wire may have a substantially similar length and profile as the core wire discussed above, or the core wire may have different dimensions than previously discussed.

The core wire may have a proximal portion and a distal portion. The proximal portion may comprise a uniform diameter and the distal portion may comprise a tapered diameter which reduces linearly or non-linearly over its length. For example, starting from a distal end of the proximal portion, the outer diameter of the core wire may be tapered from 0.035 inches to 0.021 inches to 0.018 inches to 0.014 inches at its distal tip. The distal tip may be tapered and/or flattened to accommodate a radiopaque coil as discussed below.

The proximal portion may comprise a majority of a length of the core wire, and the distal portion may comprise a minority of the length of the core wire. Thus, the proximal portion may be longer than the distal portion. For example, the distal portion may comprise about 5%-20% of an overall length of the core wire.

The core wire may be composed of various materials such as Nitinol. The overall length of the distal portion of the core wire may be comprised of about 300 mm-400 mm. A distal tip of the distal portion of the core wire may comprise approximately 5%-15% of an overall length of the distal portion. For example, the distal tip of the distal portion of the core wire may comprise about 20-30 mm.

One or more grooves may be cut into the distal portion of the core wire. The one or more grooves may extend for an entire length of the distal portion of the core wire, including its distal tip. However, the one or more grooves may instead extend for only a portion of the entire length of the distal portion of the core wire. For example, the one or more grooves may terminate at or prior to the start of the distal tip of the distal portion of the core wire.

The one or more grooves may comprise one or more coiled grooves. The one or more coiled grooves may be continuous and/or discontinuous. The one or more coiled grooves may comprise a first coiled groove and a second coiled grooves. The coiled grooves may be formed by sweep cutting into the body of the core wire. The first coiled groove may be wound in a first direction and the second coiled groove may be wound in a second direction, with the first direction being opposite to the second direction. For example, the first coiled groove may be cut in a clockwise direction and the second coiled groove may be cut in a counterclockwise direction. The first and second coiled grooves may overlap or intersect at certain intervals to form an X-pattern when intersecting.

The one or more grooves may be non-coiled or take on any other non-coiled shape. As an example, the one or more grooves may comprise a plurality of separate circular groove shapes or ringed grooves formed along all or a portion of the core wire, including all or a portion of the distal portion of the core wire. The one or more grooves may be positioned along or on the distal tip.

A radiopaque marker may be affixed to the distal tip of the core wire. The radiopaque marker may comprise, e.g., a coiled wire and/or a marker band. The radiopaque marker may be composed of various radiopaque materials, such as platinum tungsten. For example, the radiopaque marker may be comprised of a platinum tungsten wire which is coiled around the distal tip of the core wire.

The distal tip of the core wire may include a cap or other blunted, rounded, or curved projection which prevents the distal tip of the core wire from terminating in a point or pointy end which could potentially penetrate tissue. The distal tip may itself be rounded or blunted, without a separate cap being necessary.

The example embodiments shown in the figures and described herein may be designed to improve torqueability, shapeability, and resilience (i.e., the ability to retain its original shape). The example embodiments may have a shapeable tip for introduction into the human body and to provide a torqueability advantage during navigation through tortuous anatomies. A crossing profile and variable stiffness profile may contribute to the torqueability of the guidewire system.

Example embodiments may utilize a pair of oppositely wound coils which provide torque in more than one direction. A distal tip of the guidewire system may be pre-shaped by an operator to suit different anatomies. A shaping mandrel may be provided with the guidewire system to aid in shaping the distal tip to a desired shape or curvature.

Specific example embodiments are described further below. However, it should be understood that any of the features from any of the embodiments can be mixed and matched with each other in any combination. Hence, the present disclosure should not be restricted to only these embodiments, but any broader combination thereof.

The figures illustrate example embodiments of a guidewire system suitable for navigation through tortuous vessels to reach a distant target location. For example, the guidewire system may be suitable for navigation through the cerebral hemisphere.

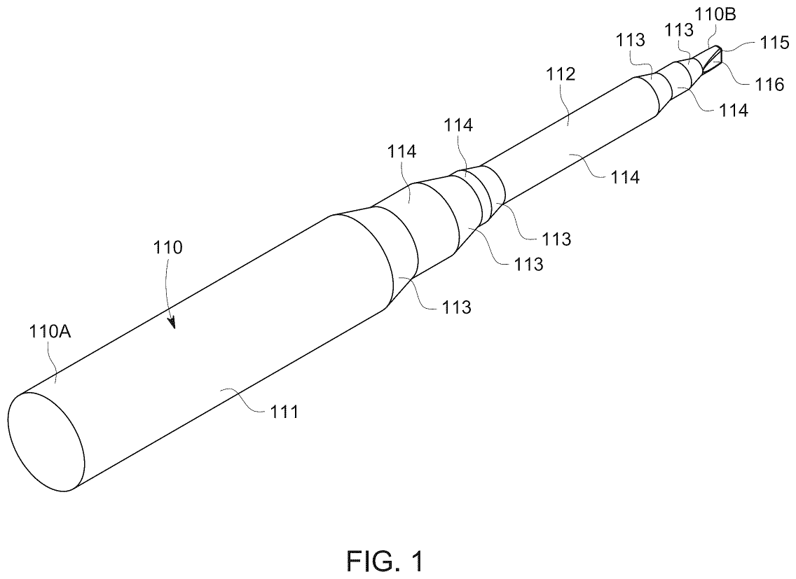

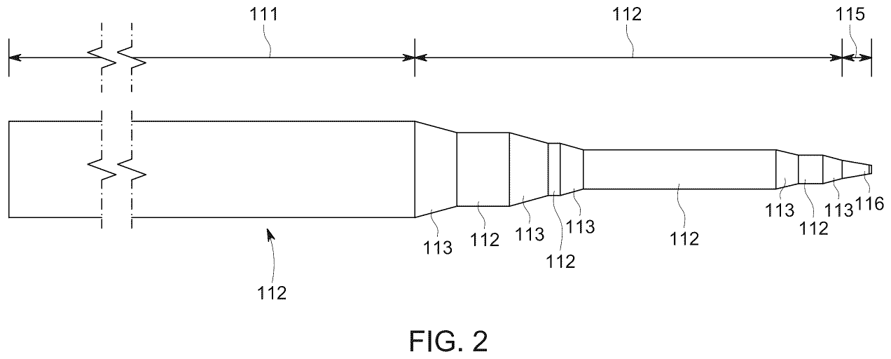

FIGS. 1 and 2 illustrate an example embodiment of a core wire 110 of a guidewire system 100 from a perspective view and a side view, respectively. As shown in FIG. 1, the core wire 110 may comprise a proximal portion 111 including its proximal end 110A and a distal portion 112 including its distal end 110B. The proximal portion 111 of the core wire 110 may comprise a uniform outer diameter. In some example embodiments, the distal portion 112 of the core wire 110 may comprise an overall tapered outer diameter including tapered diameter segments 113. In some example embodiments, the distal portion 112 of the core wire 110 may also comprise one or more uniform diameter segments 114.

The respective lengths of the proximal portion 111 and the distal portion 112 of the core wire 110 may vary in different embodiments. Generally, the proximal portion 111 may comprise at least about half of an entire length of the core wire 110. In some embodiments, the proximal portion 111 may comprise at least about 75% of an entire length of the core wire 110. In yet other embodiments, the proximal portion 111 may comprise at least about 85% of an entire length of the core wire 110. Thus, the distal portion 112 of the core wire 110 may comprise between about 15%-50% of the entire length of the core wire 110 in some embodiments. However, it should be appreciated that the distal portion 112 in certain embodiments may comprise about less than 15%, or about greater than 50%, of the entire length of the core wire 110.

By way of non-limiting examples, certain dimensions are discussed herein. However, it should be appreciated that such dimensions are merely for exemplary purposes. Thus, the scope of the present should not be construed as limited to any particular dimensions disclosed herein.

In an example embodiment, an entire length of the core wire 110, and thus the overall guidewire system 100, may comprise about 2000 mm. In some embodiments, the length of the core wire 110 may be between about 1500 mm 2500 mm. In some embodiments, of the entire length of the core wire 110, approximately 60%-80% (e.g., 70%) of the length of the core wire 110 may comprise a coated wire, such as a PTFE coated wire. The remaining length of the core wire 110, approximately 20%-40% (e.g., 30%) of the length of the core wire 110 may comprise a Nitinol core wire.

In an example embodiment such as shown in FIGS. 1-2, the proximal portion 111 of the core wire 110 may be defined as being approximately 80% of its overall length. However, in some embodiments, the proximal portion 111 may comprise greater or less than 80% of the overall length of the core wire 110. Similarly, the distal portion 112 of the core wire 110 may be defined as being approximately 20% of its overall length. However, in some embodiments, the distal portion 112 may comprise greater or less than 20% of the overall length of the core wire 110. For example, in the embodiment shown in FIGS. 1-2 the proximal portion 111 may comprise about 1600 mm and the distal portion 112 may comprise about 400 mm.

As further illustrated in FIGS. 1-2, in some embodiments, the distal portion 112 may comprise a ground core wire, such as ground Nitinol, which reduces in outer diameter (i.e., tapers) towards its distal end 110B. The use of a ground core wire 110 at the distal portion 112 may aid in both torqueability and shapeability, which improves navigation of the core wire 110 and overall guidewire system 100.

The rate and degree of tapered distal portion 112 may vary in different embodiments. In the embodiment illustrated in FIG. 2, the distal portion 112 may comprise both a plurality of tapered diameter segments 113 and a plurality of uniform diameter segments 114. In some embodiments, the tapered diameter segments 113 may be staggered with the uniform diameter segments 114 such that each tapered diameter segment 113 is adjacent to a uniform diameter segment 114, and vice versa. Put differently, the tapered diameter segments 113 and uniform diameter segments 114 may alternate along at least a portion of the length of the distal portion 112 of the core wire 110.

Continuing to reference FIG. 2, the length of the respective tapered and uniform diameter segments 113, 114 may vary across the length of the distal portion 112 of the core wire 110. For example, uniform diameter segments 114 may be comprised of lengths of 105 mm, 40 mm, 205 mm, and 40 mm in some embodiments, with the remaining length of the distal portion 112 comprising tapered diameter segments 113. Thus, in some embodiments, the uniform diameter segments 114 may comprise approximately 80%-90% of the length of the distal portion 112 of the core wire 110, and the remaining 10%-20% of the length of the distal portion 112 may be comprised of tapered diameter segments 113. However, in some embodiments, up to about 100% of the length of the distal portion 112 may comprise only tapered diameter segments 113, without any uniform diameter segments 114.

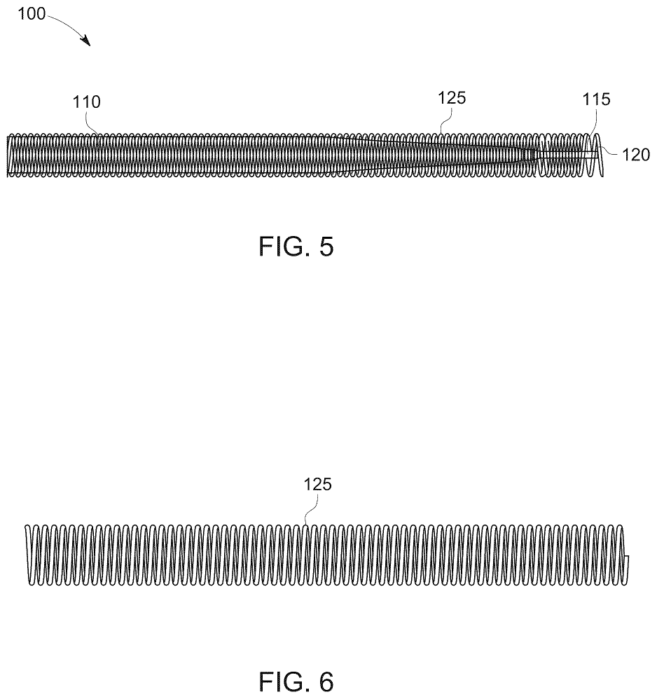

As shown in FIGS. 1-2, the distal tip 115 of the core wire 110 may comprise a flattened portion such as a paddle 116. The length of the paddle 116 may be between about 5 mm-11 mm in an example embodiment, though its length could be less than 5 mm or greater than 11 mm in certain embodiments. The paddle 116 may perform a number of functions. For example, the paddle 116 may accommodate both the inner and outer coils (discussed below with respect to FIGS. 5-10). In some embodiments, the paddle 116 may maintain a minimum annular region between itself and the inner coil. In some embodiments, the paddle 116 may allow the entry of the polymer jacket between the coils and the core wire. Accordingly, in some embodiments, the flattened paddle 16 can enhance vascular access by transmitting torque efficiently from proximal end to distal tip of the guidewire and can further facilitate better control during guidewire tracking.

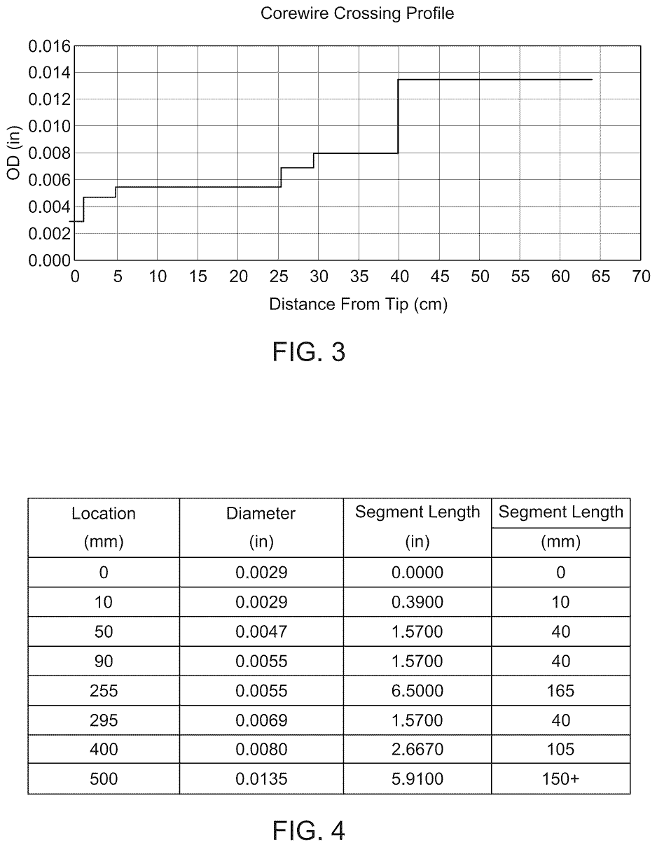

FIG. 3 illustrates a graph of an example embodiment of a core wire 110 crossing profile illustrating how the outer diameter of the distal portion 112 of the core wire 110 may taper towards its distal end 110B. Note that the x-axis of the graph illustrated in FIG. 3 lists distance from tip (i.e., distance from the distal end 110B). As can be seen, the outer diameter of the distal portion 112 of the core wire 110 may tapered in steps, with the uniform diameter segments 114 comprising the majority of the length of the distal portion 112 of the core wire 110.

FIG. 4 illustrates the graph of FIG. 3 in table form, with the “location” being calculated as the distance, in mm, from the distal end 110B of the core wire 110. As can be seen, the outer diameter of the distal portion 112 of the core wire 110 may taper from an initial outer diameter of about 0.0135 inches down to an ending outer diameter of about 0.0029 inches. Thus, the outer diameter of the distal portion 112 may be reduced by about between 75%-85% from its initial outer diameter to the ending outer diameter at its distal end 110B.

It should be appreciated that the values shown and described in FIGS. 3-4 are merely for exemplary purposes, and thus they should not be construed as limiting in scope. It is anticipated that different dimensions may be utilized to suit different applications or medical procedures.

FIG. 5 illustrates a side view of a guidewire system 100. In some embodiments, one or more coils 120, 125 may be affixed over the core wire 110 including the distal tip 115. The one or more coils 120, 125 may at least partially surround the core wire 110. In the embodiment shown in FIG. 5, a first coil 120 and a second coil 125 may be affixed over the core wire 110, with the first coil 120 comprising an inner coil and the second coil 125 comprising an outer coil 125 which is positioned over the first coil 120.

Various types of coils 120, 125 may be utilized. In an example embodiment, the first coil 120 may be composed of a different material than the second coil 125. For example, the first coil 120 may be composed of stainless steel and the second coil 125 may be composed of platinum tungsten.

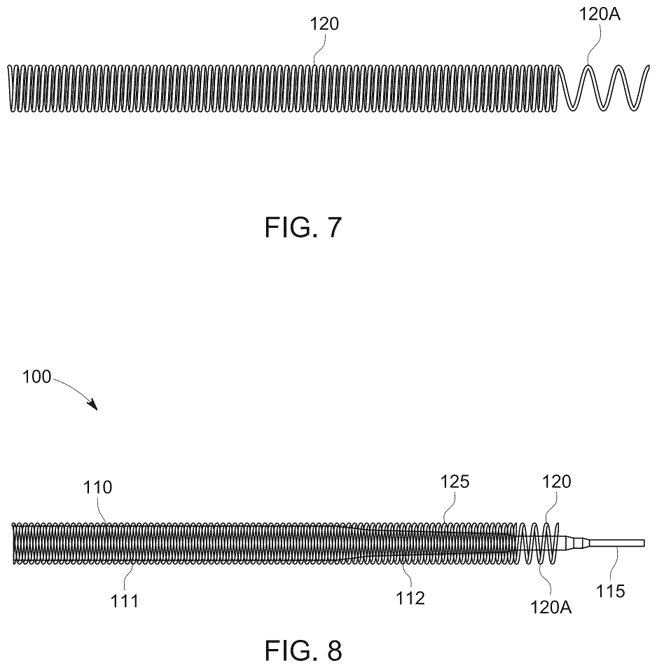

FIG. 6 illustrates side view of a second (outer) coil 125 of the guidewire system 100 in accordance with an example embodiment. FIG. 7 illustrates a side view of a first (inner) coil 120 of the guidewire system 100 in accordance with an example embodiment. In some example embodiments, the length of the first and second coils 120, 125 may be substantially similar. For example, each of the first and second coils 120, 125 may be comprised of about 250-350 mm in length. However, in some embodiments, the lengths of the first and second coils 120, 125 may be slightly different. In an example embodiment, a length of the first (inner) coil 120 may be about 280 mm and a length of the second (outer) coil 125 may be about 175 mm. Similarly, the first and second coils 120, 125 may comprise substantially the same filar dimension, such as a filar outer diameter of 0.00125 inches.

With reference to FIG. 6, the second coil 125 may comprise a uniform pitch along its overall length. For example, the length of the second coil 125 may comprise a uniform pitch of about 0.00150 inches-0.00200 inches, or about 0.00175 inches.

With reference to FIG. 7, in some embodiments, the first coil 120 may have an increased pitch along a distal portion thereof, such as between 100 mm-150 mm of its distal length. For the majority of the proximal length of the first coil 120, the coil pitch may be between about 0.00150-0.00200 inches, or about 0.00175 inches, with a proximal coil gap of between about 0.0001 inches-0.0010 inches, or about 0.0005 inches. The remaining distal length of the first coil 120 may have a gradually increased pitch, including pitches of approximately 0.005 inches, 0.008 inches, and 0.013 inches.

As shown in FIG. 5, the coils 120, 125 may be positioned over and affixed to an entire length of the core wire 110, or only a portion of the length of the core wire 110. In the example embodiment shown in FIG. 5, it can be seen that the coils 120, 125 are both affixed over the length of the core wire 110.

FIG. 8 illustrates a side view of a guidewire system 100 where coils 120, 125 are positioned over a portion of the length of the core wire 110. In the example embodiment shown in FIG. 8, it can be seen that the distal tip 115 of the core wire 110 is left exposed, and thus not covered by either of the coils 120, 125.

The direction of winding of the first and second coils 120, 125 may vary in different embodiments. In an example embodiment as shown in the figures, the first coil 120 may be wound in a first direction and the second coil 125 may be wound in a second direction, with the first direction being opposite to the second direction. By way of example, the first coil 120 may be wound in a counterclockwise direction and the second coil 125 may be wound in a clockwise direction, or vice versa. Thus, the first and second coils 120, 125 may cross each other at multiple points along the length of the core wire 110.

The manner by which the coils 120, 125 are affixed to the core wire 110 may vary in different embodiments. In an example embodiment, the respective proximal and distal sections of each of the coils 120, 125 may be soldered to the core wire 110 and then encapsulated in a polymer jacket as described herein.

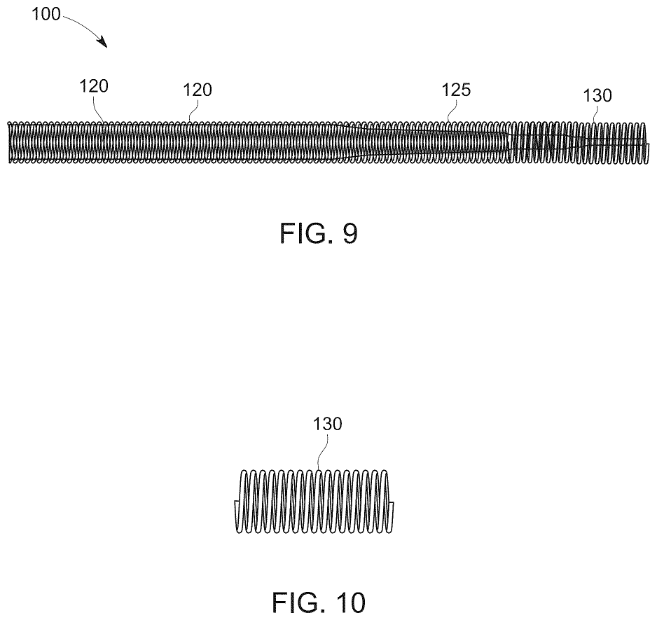

FIG. 9 illustrates a side view of a guidewire system 100 that includes three coils. In the example embodiment shown in FIG. 9, in addition to the first and second coils 120, 125, a third coil 130 may be affixed over the distal tip 115 of the core wire 110. The third coil 130 may function as an extension coil which is axial with respect to the first and second coils 120, 125. In some embodiments, the third coil 130 may overlap both the first and second coils 120, 125 at least partially. Generally, the third coil 130 will be substantially shorter in length than the first and second coils 120, 125. For example, the length of the third coil 130 may be approximately 10% of the length of the first and/or second coils 120, 125. As a more specific example, the length of the third coil 130 may be between about 20 mm-30 mm.

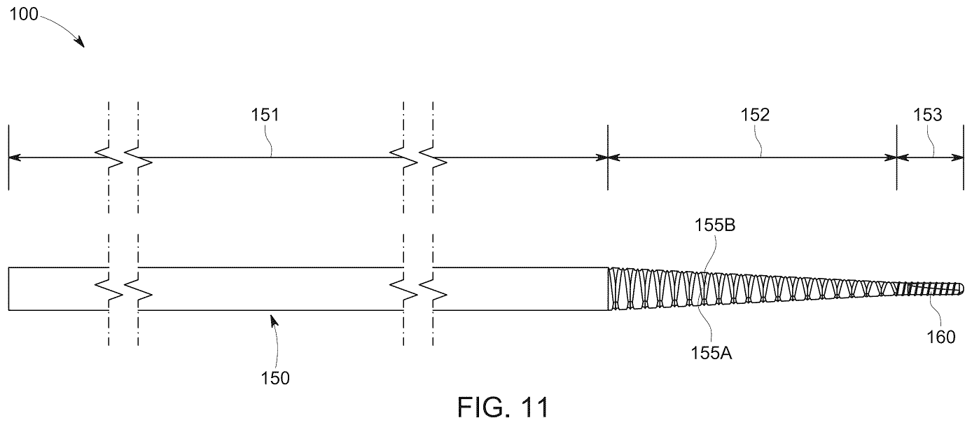

FIG. 10 illustrates a side view of an example embodiment of a third (extension) coil 130. In some embodiments, the third coil 130 may be composed of the same material as the second coil 125, such as platinum tungsten. In some embodiments, however, the third coil 130 may be the same material as the first coil 120, or may be a different material than the first and second coils 120, 125. The third coil 130 may have the same pitch as the first and/or second coils 120, 125, or may have a different pitch than the first and/or second coils 120, 125. The third coil 130 may comprise a uniform pitch across its length such as shown in FIG. 10 or may have a variable pitch.

As previously discussed, at least a portion of the core wire 110, including the one or more coils 120, 125, 130, may be encapsulated in a polymer jacket, which aids in, e.g., holding the core wire 110 and coils 120, 125, 130 together. The manner by which the polymer jacket is applied may vary in different embodiments. In an example embodiment, the polymer jacket may be applied by dip coating and/or use of a shrink tube. The reduced diameter of the distal tip 115 of the core wire 110 may aid in the polymer jacket penetrating between the coils 120, 125, 130. Various materials may be utilized for the polymer jacket, including but not limited to polyurethane.

FIGS. 11-15 illustrate example embodiments of a guidewire system 100 in which one or more grooves 155A, 155B may be cut into a core wire 150 so as to perform the same function of improving torqueability and shapeability as the separate coils 120, 125, 130 of the previously discussed example embodiments. Thus, the example embodiments shown in FIGS. 11-15 illustrate a core wire 110 with integrated, rather than discrete, coils cut into the core wire 110.

The one or more grooves 155A, 155B may comprise coiled or non-coiled grooves. In an example embodiment, the one or more grooves 155A, 155B may comprise a non-coiled shape, such as separate circular or ring-shaped grooves which are spatially separated along all or a portion of a length of the core wire 110. The one or more grooves 155A, 155B may be continuous and/or discontinuous.

FIG. 11 illustrates a side view of an example embodiment of a core wire 150 of a guide wire system 100 into which coiled grooves 155A, 155B have been cut. As shown in FIG. 11, the core wire 150 may comprise a proximal portion 151 and a distal portion 152 including a distal tip 153. The proximal portion 151 may comprise a majority of the overall length of the core wire 150.

In an example embodiment, the length of the proximal portion 151 of the core wire 150 may be defined as being approximately 80% of its overall length. However, in some embodiments, the length of the proximal portion 151 may comprise greater or less than 80% of the overall length of the core wire 150. Similarly, the length of the distal portion 152 of the core wire 150 may be defined as being approximately 20% of its overall length. However, in some embodiments, the length of the distal portion 152 may comprise greater or less than 20% of the overall length of the core wire 150.

By way of example, the length of the distal portion 152 of the core wire 150 may comprise between approximately 340 mm-400 mm. The distal tip 153 of the distal portion 152 of the core wire 150 may comprise between approximately 5%-10% of an overall length of the distal portion 152. In an example embodiment, the length of the distal tip 153 may comprise between about 20 mm-30 mm of the overall length of the distal portion 152.

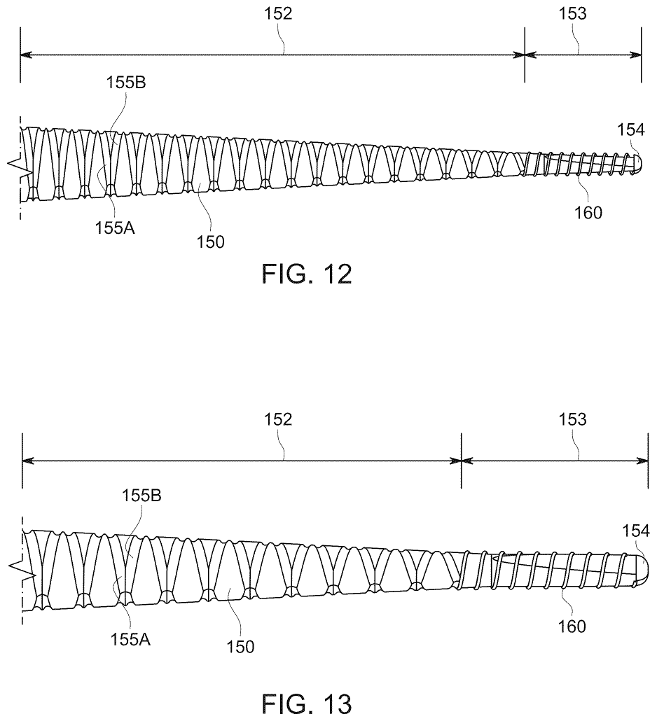

As shown in the figures, the distal portion 152 of the core wire 150 may be tapered so as to reduce in diameter distally. By way of example, the outer diameter of the distal portion 152 of the core wire 150 may reduce from about 0.035 inches to about 0.014 inches. To prevent the distal tip 153 from terminating in a point which can penetrate tissues and thus cause injuries or complications, a cap 154 may be affixed over the distal tip 153 of the core wire 150 such as shown in FIGS. 12-13. Alternatively, the distal tip 153 may itself be blunted without a separate cap 154 being necessary.

FIGS. 12-14 illustrate a view (side view and sectional view) of a distal tip 153 of a core wire 150 of a guidewire system 100 where one or more coiled grooves 155A, 155B may be cut into the core wire 150. In an example embodiment, the one or more coiled grooves 155A, 155B may be cut into at least a portion of the distal portion 152 of the core wire 150. In the example embodiment shown in FIGS. 12-13 the coiled grooves 155A, 155B may not extend into the distal tip 153 of the distal portion 152, however, in some embodiments, the coiled grooves 155A, 155B may also extend into the distal tip 153.

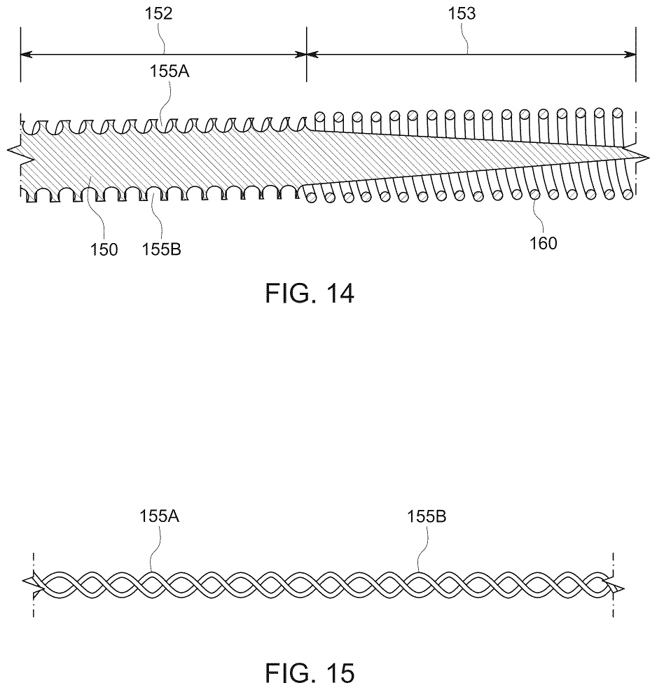

FIGS. 12-14 illustrate that a radiopaque marker 160, such as a coiled wire composed at least partially of a radiopaque material, may be positioned along all or part of the length of the distal portion 153. FIGS. 12-13 illustrate an example embodiment in which the radiopaque marker 160 may be more tightly wound around at least a portion of the distal portion 153. FIG. 14 illustrates an example embodiment in which a radiopaque marker 160 may be more loosely wound around at least a portion of the distal portion 153.

In an example embodiment, a first coiled groove 155A may be cut into the core wire 150 in a first direction and a second coiled groove 155B may be cut into the core wire 150 in a second direction, with the first direction being opposite to the second direction. By way of example, the first coiled groove 155A may be cut in a clockwise orientation and the second coiled groove 155B may be cut in a counterclockwise direction, or vice versa. Thus, the first and second coiled grooves 155A, 155B may intersect at certain points to form an X-pattern.

FIG. 15 illustrates an example embodiment of a sweep cut pattern for the opposing first and second coiled grooves 155A, 155B. The manner by which the coiled grooves 155A, 155B are formed in the core wire 150 may vary in different embodiments. By way of example, the coiled grooves 155A, 155B may be formed by performing opposing sweep cuts into the body of the core wire 150 to form the grooves therein. The core wire crossing profile may be achieved by making a variable depth of sweep cuts across the distal portion 152 of the core wire 150 to provide the desired guidewire performance requirements.

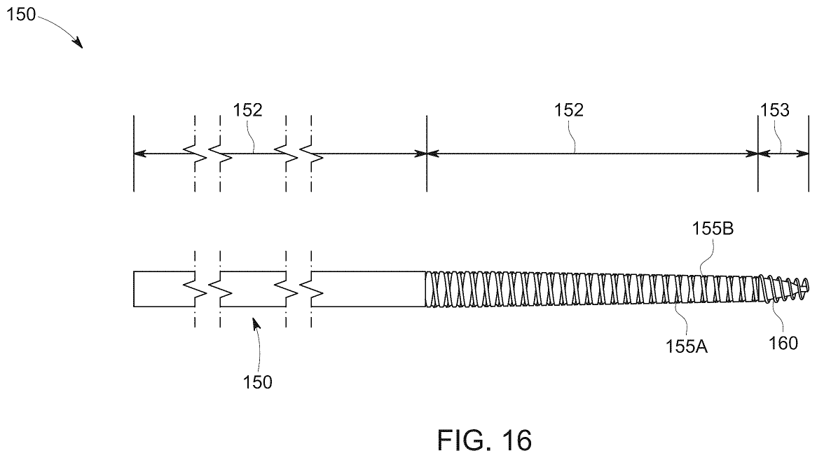

FIG. 16 illustrates a side view of a guidewire system with a radiopaque marker 160 affixed over a portion of the distal portion of the core wire 150. In example embodiments such as shown in FIGS. 11-14 and 16, a separate radiopaque marker 160 may be affixed over at least a portion of the distal portion 152 of the core wire 150. In the embodiments shown in FIGS. 11-14 and 16, it can be seen that the radiopaque marker 160 may comprise a wire which is coiled around the distal tip 153 of the core wire 150. The radiopaque marker 160 may in other embodiments comprise a marker band or the like, however. The radiopaque marker 160 may be composed of various radiopaque materials, such as but not limited to platinum tungsten.

In use, the improved shapeability and torqueability of the various example embodiments of guidewire systems 100 shown and/or described herein may ease navigation to a distal anatomy through tortuous vasculatures. The tip of the core wire may be pre-shaped by a physician prior to use to suit the planned path to a target location. In some embodiments, a separate mandrel may be provided with the guidewire system 100 to ease pre-shaping of the tip of the core wire prior to use.

### CLAUSES

Exemplary embodiments are set out in the following numbered clauses:

Clause 1. A guidewire system, comprising: an elongated body including a proximal portion and a distal portion, wherein the proximal portion is comprised of a uniform diameter and wherein the distal portion is comprised of a tapering diameter; a first coil means for imparting torqueability and/or shapeability cut into the distal portion of the elongated body; a second coil means for imparting torqueability and/or shapeability cut into the distal portion of the elongated body; and wherein the first coil means is oriented in a first direction, wherein the second coil means is oriented in a second direction, and wherein the first direction is opposite to the second direction.

Clause 2. The guidewire system of clause 1, wherein the first coil means is comprised of a first groove cut into the core wire and wherein the second coil means is comprised of a second groove cut into the core wire.

Clause 3. The guidewire system of clause 1, wherein the first coil means is comprised of a first coiled wire and wherein the second coil means is comprised of a second coiled wire.

Clause 4. The guidewire system of clause 3, wherein the first coiled wire is an outer coil and wherein the second coiled wire is an inner coil.

Clause 5. The guidewire system of clauses 3 or 4, wherein the first coiled wire is composed of stainless steel and wherein the second coiled wire is composed of platinum tungsten.

Clause 6. The guidewire system of any of the preceding clauses, further comprising a radiopaque marker affixed to a distal tip of the distal portion of the elongated body.

Clause 7. The guidewire system of clause 7, wherein the radiopaque marker is comprised of a coiled wire.

Clause 8. The guidewire system of clause 7, wherein the radiopaque marker is comprised of a marker band.

Clause 9. The guidewire system of any of the preceding clauses, further comprising a polymer jacket encapsulating the elongated body, the first coil means, and the second coil means.

Clause 10. The guidewire system of any of the preceding clauses, wherein a length of the first coil means is substantially similar to a length of the second coil means.

Clause 11. The guidewire system of any of the preceding clauses, wherein the first direction is clockwise and wherein the second direction is counterclockwise.

Clause 12. The guidewire system of any of the preceding clauses, wherein the elongated body is comprised of a core wire.

Clause 13. The guidewire system of any of the preceding clauses, wherein the elongated body is composed of Nitinol.

Clause 14. The guidewire system of any of the preceding clauses, wherein the distal portion is comprised of a plurality of uniform diameter segments and a plurality of tapering diameter segments.

Clause 15. The guidewire system of any of the preceding clauses, wherein the uniform diameter segments and the tapering diameter segments are staggered.

Clause 16. The guidewire system of any of the preceding clauses, wherein each of the plurality of tapered diameter segments is adjacent to one of the plurality of uniform diameter segments.

Clause 17. The guidewire system of any of the preceding clauses, wherein a filar dimension of the first coil means is substantially similar to a filar dimension of the second coil means.

Clause 18. The guidewire system of any of the preceding clauses, wherein a length of the first coil means is substantially similar to a length of the second coil means.

Clause 19. The guidewire system of any of the preceding clauses, wherein a pitch of the first coil means is uniform.

Clause 20. The guidewire system of any of the preceding clauses, wherein a pitch of the second coil means is variable.

Clause 21. The guidewire system of any of the preceding clauses, wherein a pitch of the second coil means increased towards its distal end.

Clause 22. The guidewire system of any of the preceding clauses, wherein a distal tip of the elongated body is flattened.

Clause 23. The guidewire system of any of the preceding clauses, wherein a distal tip of the elongated body comprises a paddle.

Clause 24. The guidewire system of any of the preceding clauses, further comprising a mandrel for pre-shaping a distal tip of the elongated body.

Clause 25. The guidewire system of any of the preceding clauses, further comprising a cap affixed to a distal tip of the elongated body.

Clause 26. The guidewire system of any of the preceding clauses, wherein the first coiled means is composed of stainless steel.

Clause 27. The guidewire system of any of the preceding clauses, wherein the second coiled means is composed of platinum tungsten.

Clause 28. A guidewire, comprising: an elongated body including a proximal portion and a distal portion, wherein the proximal portion is comprised of a uniform diameter, and wherein the distal portion is comprised of only a plurality of tapered diameter segments.

Clause 29. The guidewire of clause 28, wherein each of the plurality of tapered diameter segments reduce in diameter towards a distal end of the elongated body.

Although the disclosure has been described in terms of particular embodiments and applications, one of ordinary skill in the art, in light of this teaching, can generate additional embodiments and modifications without departing from the spirit of or exceeding the scope of the claimed. Accordingly, it is to be understood that the drawings and descriptions herein are proffered by way of example to facilitate comprehension of the and should not be construed to limit the scope thereof.

1. A guidewire, comprising:
an elongated body including a proximal portion and a distal portion;
wherein the proximal portion is comprised of a uniform diameter;
wherein the distal portion is comprised of a tapering diameter; and,
a first continuous groove cut into an elongated length of the distal portion of the elongated body.

2. The guidewire of claim 1, wherein the distal portion comprises no more than one third of a length of the elongated body.

3. The guidewire of claim 1, wherein the first groove is comprised of a clockwise sweep cut pattern.

4. The guidewire of claim 1, further comprising a second groove cut into the distal portion of the elongated body.

5. The guidewire of claim 4, wherein the first groove and the second groove overlap.

6. The guidewire of claim 4, wherein the first groove is cut in a first direction, wherein the second groove is cut in a second direction, and wherein the first direction is opposite to the second direction.

7. The guidewire of claim 4, wherein the first groove is comprised of a clockwise sweep cut pattern and wherein the second groove is comprised of a counterclockwise sweep cut pattern.

8. The guidewire of claim 1, wherein a distal end of the elongated body includes a flattened distal tip.

9. The guidewire of claim 1, further comprising a wire coiled around at least a portion of the distal portion of the elongated body.

10. The guidewire of claim 1, wherein the first groove is comprised of a first coiled groove.

11. A guidewire, comprising:
an elongated body including a proximal portion and a distal portion;
wherein the proximal portion is comprised of a uniform diameter;
wherein the distal portion is comprised of a plurality of tapered diameter segments.

12. The guidewire of claim 11, wherein the distal portion is further comprised of a plurality of uniform diameter segments.

13. The guidewire of claim 12, wherein each of the plurality of tapered diameter segments is adjacent to one of the plurality of uniform diameter segments.

14. The guidewire of claim 11, wherein the proximal portion is comprised of between at least half of a length of the elongated body.

15. The guidewire of claim 11, further comprising a first coiled wire and a second coiled wire, the first and second coiled wires each being positioned over at least a portion of the elongated body.

16. The guidewire of claim 15, wherein the first coiled wire is comprised of an inner coiled wire and wherein the second coiled wire is comprised of an outer coiled wire.

17. The guidewire of claim 15, further comprising a third coiled wire positioned over a distal tip of the elongated body.

18. The guidewire of claim 15, further comprising a polymer jacket encapsulating the elongated body, the first coiled wire, and the second coiled wire.

19. The guidewire of claim 15, wherein the first coiled wire is wound in a first direction, wherein the second coiled wire is wound in a second direction, and wherein the first direction is opposite to the second direction.

20. A guidewire, comprising:
an elongated body including a proximal portion and a distal portion;
wherein the proximal portion is comprised of a uniform diameter;
wherein the distal portion is comprised of a plurality of tapered diameter regions and a plurality of uniform diameter regions;
an inner coil affixed to at least partially surround the distal portion of the elongated body; and
an outer coil affixed to at least partially surround the distal portion of the elongated body, wherein the outer coil is positioned over the inner coil; and,
wherein the inner coil is wound in a first direction, wherein the outer coil is wound in a second direction, and wherein the first direction is opposite to the second direction.

21-22. (canceled)
